(19) United States
(12) Patent Application Publication
(10) Pub. No.: US 2008/0097367 A1
(43) Pub. Date: Apr. 24, 2008

(54) DIAPER

(76) Inventor: Jeong Wook Jeon, Pyungtaek-si (KR)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/874,498

(22) Filed: Oct. 18, 2007

(30) Foreign Application Priority Data
Oct. 23, 2006 (KR) 10-2006-0102669
Oct. 1, 2007 (KR) 10-2007-0098589

(51) Int. Cl. A61F 13/15

(52) U.S. Cl. 604/385.23; 604/385.01; 604/385.24

(57) ABSTRACT

Disclosed herein is an expandable diaper. The present invention provide a new type of diaper which minimizes the area to be in contact with skin of a wearer, maximizes the absorption of excrement, and preventing the excrement from sticking to the skin of the wearer, by forming a portion of the diaper to be expandable or forming a wrinkled portion on the diaper, thus preventing various skin diseases even though the wearer puts on the diaper for a long time, and protecting the skin of the wearer from various skin troubles due to the excrement.

### CROSS-REFERENCE TO RELATED APPLICATION

This application claims the benefit of Korean Patent Application No. 10-2007-0098589, filed on Oct. 1, 2007 and No. 10-2006-0102669, filed on Oct. 23, 2006, the entire disclosure of which is hereby incorporated by reference.

### BACKGROUND OF THE INVENTION

1. Field of the Invention

The present invention relates to a diaper and, more particularly, to a new type of diaper which minimizes the area to be in contact with skin of a wearer and maximizes the absorption of excrement by forming a portion of the diaper to be expandable or forming a wrinkled portion on the diaper, thus preventing skin diseases such as skin maceration, skin pressure, diaper rash, and the like due to the excrement.

2. Description of Related Art

In general, diapers are disposable products to meet the social demands in the pursuit of convenience and are usefully applied to infants who are not grown up enough to go to stool by themselves, old people or patients with special diseases, thus preventing excrement from leaking.

Such a disposable diaper comprises a front sheet made of a liquid permeable material through which liquid excrement permeates, a back sheet made of a liquid non-permeable material preventing the permeating liquid from leaking, and an absorbent member formed by adding a super absorbent polymer to natural pulp and disposed between the front sheet and the back sheet to rapidly absorb the excrement.

Moreover, the disposable diaper further comprises elastic members for increasing wearability and adherence to prevent excrement from leaking and fastening means for fastening the diaper.

With the use of high performance absorbents, the thickness of the disposable diapers is gradually reduced. Moreover, the disposable diapers are fabricated using high quality materials such as a front sheet made of all cotton or a material providing an equivalent feeling and a porous back sheet.

Accordingly, with the increase in the functionality, the capacity of receiving the excrement of a wearer and the wearing time are increased more and more in view of that the infant wears do not feel and express the wetness readily.

However, when wearing the disposable diaper for a long time, skin maceration may be induced by skin irritation and friction due to pH of the excrement accumulated in the diaper, and the skin maceration becomes serious by colonization and proliferation of bacteria.

Moreover, since the disposable diaper includes elastic members formed on the waist and leg regions of the diaper to prevent the excrement from leaking, when the diaper is put on the body tightly to be sealed hermetically, the body regions wrapped by the diaper are cut off from the outside, and thus humid air increases the body temperature to cause skin diseases such as pruritus, miliaria, and the like.

Furthermore, since the infants incapable of moving their bodies are only placed in their beds, their bodies are irritated by the elastic members having relatively rough surfaces, and thus skin diseases may be caused on their backs due to the stuffy diaper.

Especially, when the disposable diaper put on an infant or a patient to take care of the body wastes thereof is left for a long time due to a lack of thoughtful consideration of a guardian, it causes a drop of the body temperature and skin dermatitis due to the heat of vaporization.

It is known that the skin diseases of the infant wearing the disposable diaper are caused by skin irritation due to the generation of ammonia from the excrement, skin irritation due to the growth of Staphylococcus epidermidis, physical and chemical irritation due to the disposable diaper materials, and skin irritation by excessive humidity generated during the wearing of the disposable diaper.

In order to overcome such problems, there have been various attempts to employ various structures, functional materials for skin protection, and pH regulators; however, their effects are not satisfactory.

### SUMMARY OF THE INVENTION

Accordingly, the present invention has been made in an effort to solve the above problems, and an object of the present invention is to provide a new type of diaper which minimizes the area to be in contact with skin of a wearer, maximizes the absorption of excrement, and preventing the excrement from sticking to the skin of the wearer, by forming a portion of the diaper to be expandable or forming a wrinkled portion on the diaper, thus preventing various skin diseases even though the wearer puts on the diaper for a long time, and protecting the skin of the wearer from various skin troubles due to the excrement.

In an aspect, the present invention provides a diaper comprising: a plurality of sheet layers having an absorbent layer therein, an elastic flap portion, and a band portion having fastening means, wherein the sheet layers comprise a wrinkled portion formed over a predetermined area and expanded when a weight is applied thereto to provide a wide surface area.

The wrinkled portion may be folded repeatedly in a predetermined height over a predetermined area along the longitudinal direction of the sheet layer.

Moreover, the wrinkled portion may have an indefinitely wrinkled shape formed over a predetermined area of the sheet layer Furthermore, the wrinkled portion may be formed over the entire width of the sheet layer.

In addition, the wrinkled portion may be formed over a width of about ⅓ of the middle portion of the entire width of the sheet layer.

In this case, the wrinkled portion may have a height above a thickness of the plurality of sheet layers.

In another aspect, the present invention provides a diaper comprising a plurality of sheet layers having an absorbent layer therein, an elastic flap portion, and a band portion having fastening means, wherein the sheet layers comprise an inner sheet, a back sheet, and an absorbent layer disposed therebetween, the inner sheet, the back sheet and the absorbent layer being formed of an elastic material, respectively, or the inner sheet, the back sheet and the absorbent layer being formed integrally, such that the sheet layers are expanded when a weight is applied thereto to provide a wide surface area.

Here, the sheet layers may be expandable in the horizontal direction, in the vertical direction or in both directions.

In a further aspect, the present invention provides a diaper comprising a plurality of sheet layers having an absorbent layer therein, an elastic flap portion, and a band portion having fastening means, wherein the sheet layers comprise a wrinkled absorbent layer expanded when a weight is applied thereto to provide a wide surface area.

### BRIEF DESCRIPTION OF THE DRAWINGS

FIG. 1 is a perspective view illustrating a diaper in accordance with a preferred embodiment of the present invention;

FIG. 2 is a partial side view illustrating the diaper in accordance with the preferred embodiment of the present invention;

FIG. 3 is side views illustrating the wearing state of the diaper in accordance with the preferred embodiment of the present invention;

FIG. 4 is a front view illustrating the wearing state of a diaper in accordance with another embodiment of the present invention;

FIGS. 5A to 5D are plan views illustrating various shapes of wrinkled portions of the diaper in accordance with the present invention;

FIGS. 6A and 6B are front views and side views illustrating a diaper having expandable properties in accordance with a further embodiment of the present invention; and

FIG. 7 is side views illustrating a diaper having an absorbent layer in accordance with a still another embodiment of the present invention; and

### DETAILED DESCRIPTION OF THE INVENTION

Hereinafter, preferred embodiments in accordance with the present invention will be described with reference to the accompanying drawings. The preferred embodiments are provided so that those skilled in the art can sufficiently understand the present invention, but can be modified in various forms and the scope of the present invention is not limited to the preferred embodiments.

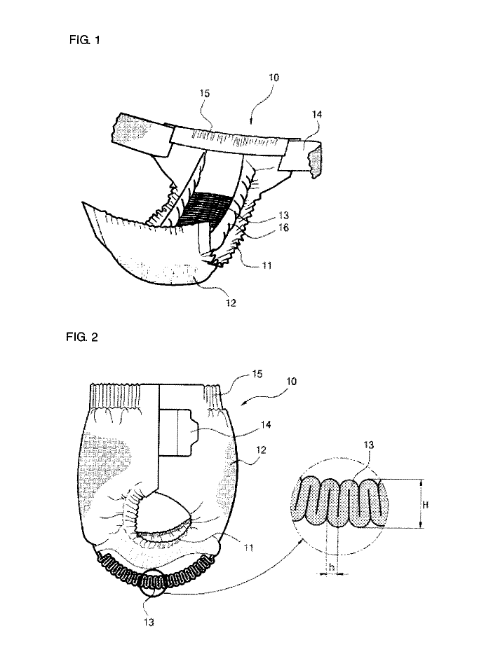

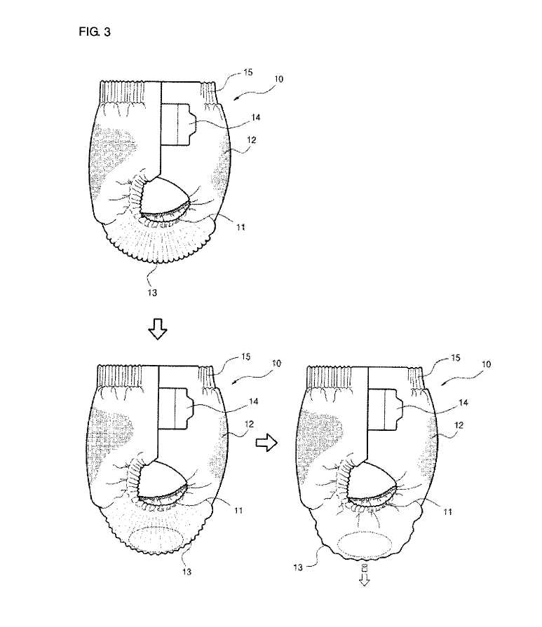

FIG. 1 is a perspective view illustrating a diaper including a wrinkled portion in accordance with a preferred embodiment of the present invention, and FIG. 2 is a partial side view illustrating the diaper including a wrinkled portion in accordance with the preferred embodiment of the present invention.

As shown in FIGS. 1 and 2, the diaper 10 of the present invention generally comprises a sheet layer 12 corresponding to a main body, an elastic flap portion 11 blocking a leg portion of a wearer, a band portion 15 providing elasticity in the width direction, fastening means 14 for fastening the diaper, and a pair of auxiliary flaps 16 having a predetermined height extending along both sides of the sheet layer 12 in the longitudinal direction.

The sheet layer 12 includes an inner sheet coming in contact with skin of a wearer, a liquid non-permeable outer sheet, and an absorbent layer including high absorbent resin disposed between the inner and outer sheets.

Especially, a wrinkled portion 13 is formed on the sheet layer to be expanded by the weight of excrement, thus preventing the skin of the infant from being in contact with the excrement.

Such a wrinkled portion 13 is folded repeatedly in a predetermined height over a predetermined area along the longitudinal direction of the diaper and, preferably, formed over the entire width of the sheet layer 12 and over a length of about ⅓ of the entire length of the sheet layer 12.

The width and length of the wrinkled portion 13 may be adjusted properly according to the size of the diaper, the age of the wearer, and the like.

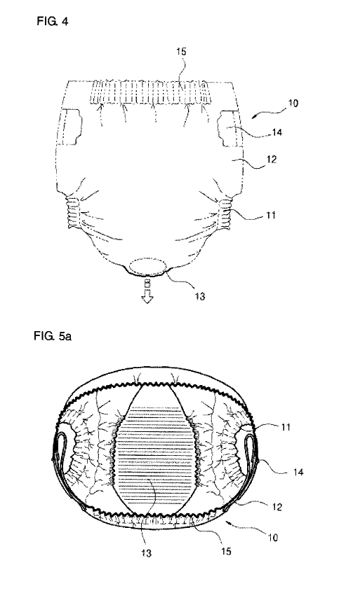

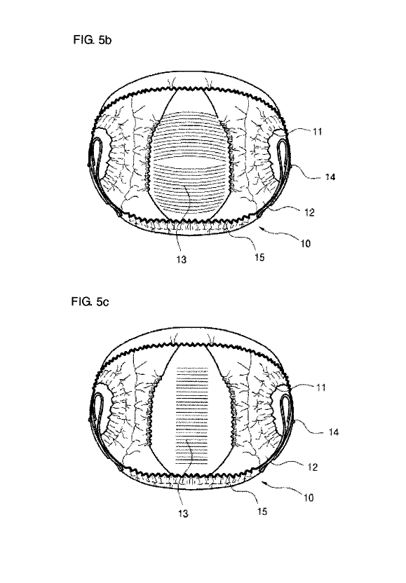

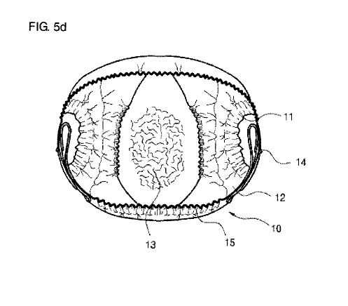

Meanwhile, as shown in FIG. 5C illustrating another embodiment of the present invention, the wrinkled portion 13 may be formed over a length of about ⅓ of the entire length of the sheet layer 12 and over a width of about ⅓ of the middle portion of the entire width of the sheet layer 12. In this case, as illustrated in FIG. 4, only the middle width portion of the sheet layer 12 is expanded downward during the excretion, thus maximizing the absorption of the excrement.

Moreover, the wrinkled portion 13 may have a shape that crosses the width direction of the sheet layer 12 in straight lines as shown in FIGS. 5A and 5C, or a shape that crosses the width direction of the sheet layer 12 in curved lines as shown in FIG. 5B.

Furthermore, as shown in FIG. 5D illustrating a further embodiment of the present invention, the wrinkle portion 13 may have an indefinitely wrinkled shape formed over a predetermined area of the sheet layer 12 such as the wrinkled surface of a stuffed dumpling boiled in water. Even in this case, the wrinkled portion 13 is expanded by the weight of excrement to increase the surface area, thus obtaining the same effect.

The wrinkled portion 13 having a shape in which the overall sheet layer 12 is folded in a predetermined height like a bellows is unfolded when the weight of feces or a large amount of urine acts thereon, thus providing a sufficient surface area capable of absorbing the excrement and preventing the excrement from sticking to the skin of the infant.

Preferably, the wrinkled portion 13 may have a height H above a thickness h of the sheet layers 12. If the height of the wrinkled portion 13 is out of the above range, the volume of the sheet layer 12 is increased and thus the effect is unsatisfactory.

Meanwhile, the wrinkled portion 13 is expanded by the weight of the excrement to protect the skin of the infant and, at the same time, minimizes the contact area with the skin in a normal wearing state.

That is, with the concave-convex surface of the wrinkled portion 13, the area in contact with the skin becomes a kind of point contacts and, accordingly, predetermined spaces are defined between the skin and the wrinkled portion 13, thus providing ventilation. Consequently, it is possible to prevent skin diseases such as skin maceration and diaper rash even though the wearer puts on the diaper for a long time.

FIG. 3 is side views illustrating the wearing state of the diaper in accordance with the preferred embodiment of the present invention.

With reference to FIG. 3, the wearing order will be described as follows. A guardian places the hips of an infant lying on its back on the unfolded diaper 10, the front portion with respect to the wrinkled portion 13 is bent to be placed on the abdominal region of the infant, and the fastening portions 14 of the rear portion of the diaper are fastened on the outer surface of the rear portion of the diaper, thus completing the wearing of the diaper.

Accordingly, when the excrement such as feces is accumulated in the region of the wrinkled portion 13, the wrinkled portion 13 is expanded by the weight of the excrement and thus the surface area of the sheet layer 12 is widened to rapidly absorb a large amount of excrement and prevent the excrement from sticking to the skin of the infant.

Moreover, in case of urine, when the amount of urine absorbed in the absorbent layer is increased, the wrinkled portion 13 is expanded by the weight of the urine and thus the sheet layer 12 having the wrinkled portion 13 is expanded and separated from the skin of the infant, thus protecting the skin of the infant from the wet diaper for a long time.

Furthermore, with the expanded wrinkled portion 13 of the diaper, the guardian can readily determine whether or not the excrement is present and take necessary steps such as replacing the wet diaper with new one.

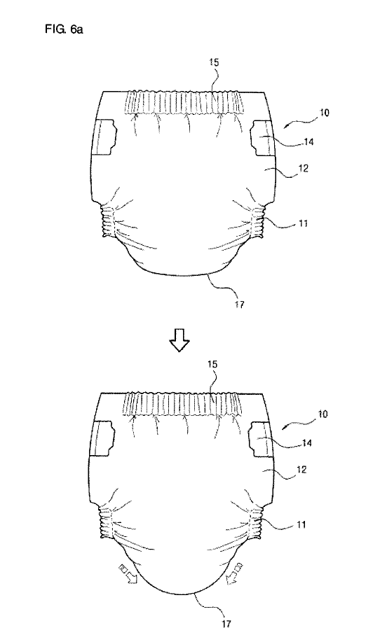

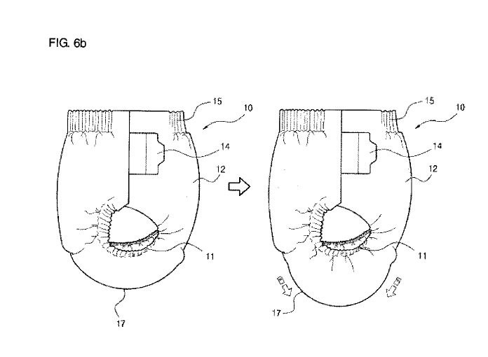

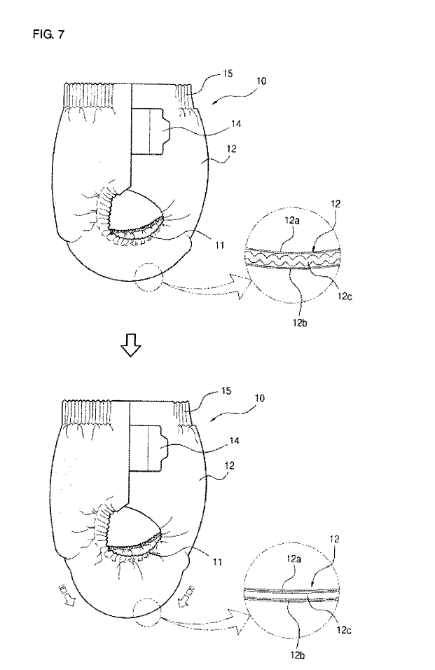

FIGS. 6A and 6B are front views and side views illustrating a diaper having expandable properties in accordance with a further embodiment of the present invention.

As illustrated in FIGS. 6A and 6B, the diaper in accordance with this embodiment of the present invention shows an expandable function using its material properties, besides the expandable properties using the wrinkled portion, differently from the above embodiments.

For this, a sheet layer 12 of the diaper includes an expandable portion 17 made of an expandable material by sewing the whole or a portion of the absorbent layer, the inner sheet and the back sheet to expand or contract naturally or artificially. Accordingly, the expandable portion 17 is expanded by the weight of feces or urine to provide a wide surface area, thus rapidly absorbing the large amount of urine and preventing the excrement from sticking to the skin of the infant.

At this time, the sheet layer 12 may be expanded in at least one direction such as the horizontal direction and the vertical direction or in both directions.

Accordingly, in a normal state, the absorbent layer is operated integrally by the inner sheet and the back sheet and, when a proper weight is given thereto, the sheet layer 12 is expanded (refer to FIGS. 6 and 7).

Here, the method of expanding the sheet layer 12 in the horizontal direction, in the vertical direction or in both directions may be adopted by appropriately selecting the weaving direction of the sheet layer 12.

Moreover, auxiliary means formed of an elastic material such as shape memory alloy or synthetic resin are provided on both sides of the sheet layer 12 or on the portion that meets the flap portion 11 in the horizontal direction of the sheet layer 12 such that the sheet layer 12 may be expanded by the temperature or the weight.

FIG. 7 is side views illustrating a diaper having an absorbent layer in accordance with a still another embodiment of the present invention.

As shown in FIG. 7, the sheet layer 12 of the diaper in accordance with this embodiment of the present invention includes a wrinkled absorbent layer 12c to be unfolded, if necessary.

For this, the sheet layer 12 is fabricated in such a manner that an inner sheet 12a and a back sheet 12b are combined with each other with the wrinkled absorbent layer 12c disposed therebetween.

Accordingly, the absorbent layer 12c is unfolded by the weight of feces or urine to provide a wide surface area, thus rapidly absorbing the large amount of urine.

When the absorbent layer 12c is expanded like this, the inner sheet 12a and the rear sheet 12b are slightly expanded in terms of its material properties so as not to disturb the unfolding of the absorbent layer 12c; however, it is preferable that the inner sheet 12a and the rear sheet 12b are made of an expandable material to be expanded flexibly together with the absorbent layer 12c.

The wrinkled portion of the absorbent layer 12c may be formed in various directions such as the horizontal direction and the vertical direction and formed in a predetermined area at the bottom.

As described above, the present invention provides the diaper which minimizes the area to be in contact with skin of a wearer, maximizes the absorption of excrement, and preventing the excrement from sticking to the skin of the wearer, by forming a portion of the diaper to be expandable or forming a wrinkled portion on the diaper, thus preventing various skin diseases even though the wearer puts on the diaper for a long time, and protecting the skin of the wearer from various skin troubles due to the excrement.

Moreover, with the expansion of the wrinkled portion of the diaper, it is possible to readily check the excrement of the wearer and take necessary steps such as replacing the wet diaper with new one.

As above, preferred embodiments of the present invention have been described and illustrated, however, the present invention is not limited thereto, rather, it should be understood that various modifications and variations of the present invention can be made thereto by those skilled in the art without departing from the spirit and the technical scope of the present invention as defined by the appended claims.

1. A diaper comprising a plurality of sheet layers having an absorbent layer therein, an elastic flap portion, and a band portion having fastening means, wherein the sheet layers comprise a wrinkled portion formed over a predetermined area and expanded when a weight is applied thereto to provide a wide surface area.

2. The diaper of claim 1, wherein the wrinkled portion is folded repeatedly in a predetermined height over a predetermined area along the longitudinal direction of the sheet layer.

3. The diaper of claim 1, wherein the wrinkled portion has an indefinitely wrinkled shape formed over a predetermined area of the sheet layer

4. The diaper of claim 1, wherein the wrinkled portion is formed over the entire width of the sheet layer.

5. The diaper of claim 1, wherein the wrinkled portion is formed over a width of about ⅓ of the middle portion of the entire width of the sheet layer.

6. The diaper of claim 1, wherein the wrinkled portion has a height above a thickness of the plurality of sheet layers.

7. A diaper comprising a plurality of sheet layers having an absorbent layer therein, an elastic flap portion, and a band portion having fastening means, wherein the sheet layers comprise an inner sheet, a back sheet, and an absorbent layer disposed therebetween, the inner sheet, the back sheet and the absorbent layer being formed of an elastic material, respectively, or the inner sheet, the back sheet and the absorbent layer being formed integrally, such that the sheet layers are expanded when a weight is applied thereto to provide a wide surface area.

8. The diaper of claim 7, wherein the sheet layers are expandable in the horizontal direction, in the vertical direction or in both directions.

9. A diaper comprising a plurality of sheet layers having an absorbent layer therein, an elastic flap portion, and a band portion having fastening means, wherein the sheet layers comprise a wrinkled absorbent layer expanded when a weight is applied thereto to provide a wide surface area.
